(19) United States
(12) Patent Application Publication
(10) Pub. No.: US 2022/0193211 A1
(43) Pub. Date: Jun. 23, 2022

(54) OVEREXPRESSION OF IMMUNOPROTEASOME IN HOST CELLS FOR GENERATING ANTIGEN-PRESENTING CELLS

(71) Applicant: INTELLISTEM TECHNOLOGIES INC., Toronto (CA)

(72) Inventors: Riam Shammaa, Toronto (CA); Moutih Rafei, Vaudreuil-Dorion (CA)

(21) Appl. No.: 17/604,294

(22) PCT Filed: Apr. 17, 2020
(86) PCT No.: PCT/CA2020/050515

§ 371 Date: Oct. 15, 2021

Related U.S. Application Data
(60) Provisional application No. 62/835,678, filed on Apr. 18, 2019.
(60) Provisional application No. 62/912,331, filed on Oct. 8, 2019.

(51) Int. Cl. A61K 39/00; C12N 9/64; A61K 39/39; A61K 39/395

(52) U.S. Cl. CPC A61K 39/0011; C12N 9/6421; A61K 2039/5156; A61K 39/39; A61K 39/3955; C12Y 304/25

(57) ABSTRACT

The present disclosure concerns genetically modified host cells that express an immunoproteasome in the absence of induction by or contact with a cytokine. The genetically modified stem cells are useful, for example, for vaccine production, and identification of new target antigens.

### CROSS REFERENCE TO RELATED APPLICATION

The application claims priority from U.S. provisional application No. 62/835,678 filed on Apr. 18, 2019, and U.S. provisional application No. 62/912,331 filed on Oct. 8, 2019, the entire contents of which are incorporated herein by reference.

### TECHNOLOGICAL FIELD

The present disclosure relates to a genetically modified cells expressing enzymes for enhancing cellular immune response activities.

### BACKGROUND

Although vaccination was proven to elicit protective responses against infectious diseases in general, cancer vaccines remain unavailable.¹⁻⁴ The major divergence between cancer vaccines and those targeting infectious agents lies in the nature of the antigen (e.g. self vs. non-self respectively).¹⁻⁴ Therefore, a cancer vaccine is challenging to develop due to obstacles related to the identification of tumor-associated/specific antigens (TAAs/TSAs) capable of generating effective and persistent cytotoxic T lymphocytes (CTL) without breaking tolerance.^{3,4} In addition, elicited CTLs must express low levels of immune checkpoint receptors to avoid tumor-mediated inhibition.⁶⁻⁷ Historically, TAA-based cancer vaccines tested in the mid-90s revealed good clinical responses in a small patient subset (10-15%), which led to the conclusion that tumor immunogenicity is both patient- and tumor-specific.⁶⁻¹⁰ As a result, efforts were dedicated to understand and optimize antigen presentation rather than searching for new TAAs/TSAs.⁷⁻¹⁰

Amongst the antigen-presenting cells (APCs) tested to date, dendritic cells (DCs) are considered the most efficient at priming immune responses. ⁶⁻¹⁰ Although shown to be safe, technically feasible, and immunogenic to a certain extent, the overall clinical results were disappointing for several reasons. To start, administered DCs do not persist long enough post-injection and their migration to secondary lymphoid organs is limited if not absent.¹⁰ In addition, there is no standardized procedure for ex vivo DC preparation, which results in a plethora of protocols differing in the source/phenotype of DCs, the maturation stimulus used, the nature/procedure for antigen loading and the route of administration.^{9,10} Third, monocyte-derived DCs have a limited cross-presentation capacity, which impedes their ability to elicit good CTL responses.¹¹ Even though a new subset of DCs (CD141+XCR1⁺) capable of efficient cross-presentation was identified in humans, their trace numbers in peripheral blood (<0.1%) limited their therapeutic use.^{11,13} Fourth, it has been shown that DC numbers in cancer patients are reduced compared to healthy donors, whereas surgical resection of tumours increases their count.^{14,15} This indicates that DCs and DC precursors derived from cancer-bearing patients are inadequate for cancer immunotherapies.^{14,15}

Therefore, generating improved antigen-presenting cells as well as establishing a more abundant supply of an APC capable of bypassing the aforementioned barriers are beeded.

### SUMMARY

In accordance with an aspect, there is provided a genetically modified host cell having one or more heterologous nucleic acid molecule encoding one or more polypeptide for the expression of an enzyme having immunoproteasome activity, wherein the heterologous nucleic acid molecule allows for the digestion and/or cross-presentation of antigen protein by the genetically modified host cell.

In accordance with another aspect, a process for making vaccines is provided, the process comprising contacting the genetically modified host cell described herein with a target under a condition that promotes protein expression.

In accordance with another aspect, a method for identifying peptide antigens is provided, the method comprising: contacting the genetically modified host cell described herein with a target; collecting peptide fragments from the genetically modified host cell; and screening the collected peptide fragments to identify antigenic peptide fragments.

In accordance with another aspect, there is provided a vaccine comprising the genetically modified host cell described herein and a pharmaceutically acceptable carrier, wherein the genetically modified host cell has been pretreated with a target.

In accordance with another aspect, there is provided method of treating a patient suffering from a virus, bacteria, or parasite infection comprising administering the vaccine described herein to a patient in need thereof.

In accordance with another aspect, there is provided a method of treating a patient suffering from cancer comprising administering the vaccine described herein to a patient in need thereof, wherein the treatment is prophylactic or therapeutic.

In accordance with another aspect, there is provided use of the genetically modified host cell described herein in the manufacture of a medicament or a vaccine for the treatment or prophylactic treatment of a viral, bacterial, or parasitic infection.

In accordance with another aspect, there is provided use of the genetically modified host cell described herein in the manufacture of a medicament or a vaccine for the therapeutic treatment or prophylactic treatment of cancer.

In accordance with another aspect, there is provided a method of obtaining exosomes from the genetically modified host cell described herein, the method comprising: culturing the genetically modified host cell in a culture medium; and collecting the supernatant from the culture medium.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 shows a graphic representation of cellular activities of a mesenchymal stem cell (MSC). A) “MSC-CPr”: mesenchymal stem cell expressing constitutive proteasome (CPr). B) “MSC-IPr”: modified mesenchymal stem cell overexpressing immunoproteasome (IPr).

FIG. 2A shows a graphic schematic representing IPr cDNA construct for the cloning strategy of IPr gene subunits (β1i, β2i, β5i). Each of the subunits are separated by a 2A peptide sequence.

FIG. 2B shows western blot depicting the expression of the three subunits in 1) dendritic cells (positive control), 2) MSC-CPr, and 3) MSC-IPr.

FIG. 3 shows a phenotypic assessment of cell surface immune molecules to compare the expression levels of various immune molecules between the different MSC populations (A, B); or bone marrow-derived mature dendritic cell (DC) (C, D). A) Flow cytometry analysis of MSC-CPr and MSC-IPr. For each histogram graph, the distribution plots from top to bottom are as follows: top plot is MSC-IPr; second from top is IFNγ-primed MSCs; third from top is MSC-CPr; and bottom plot is the isotype control (MSC-IPr). B) Mean fluorescent intensity (MFI) of the flow cytometry analysis on Y-axis. Cell population on the X-axis. For each graph, left bar is MSC-CPr, middle bar is IFNγ-primed MSCs; and right bar is MSC-IPr. C) Flow cytometry analysis of DC and MSC-IPr. For each histogram graph, the distribution plots from top to bottom are as follows: top plot is isotype control (MSC-IPr); second plot from top is MSC-IPr; third plot from top is isotype control (DC); and bottom plot is DC. D) for each graph, the bars from left to right are as follows: left bar is isotype control (MSC-IPr); second bar from left is MSC-IPr; third bar from left is isotype control (DC); and right-most bar is DC. For all shown MFI, n=5/group with *P<0.05, **P<0.01 and ***P<0.001.

FIG. 4 shows a phenotypic assessment for expression of MHCII by the MSC-IPr. A) Flow cytometry analysis of MSC-CPr (“MSC”) and MSC-IPr. B) Mean fluorescent intensity (MFI) of the flow cytometry analysis on Y-axis. Cell population on the X-axis. “I-Ab” refers to the antibody against MHCII. For all shown MFI, n=5/group with *P<0.05, **P<0.01 and ***P<0.001.

FIG. 5 shows quantification of cytokines and chemokines for dendritic cells (DCs) and MSCs. A) Quantification of various cytokines secreted by DCs vs. the MSC populations by ELISA. B) Quantification of various chemokines by protein arrays using supernatant derived from DCs or the studied MSC populations. The cell populations are dendritic cells (DC), MSC-CPr (Ctl MSC), IFNγ-primed MSCs (MSCγ), and MSC-IPr. For all results shown in this figure, n=6/group with **P<0.01.

FIG. 6 shows an assessment of the antigen cross-presentation ability of MSC-IPr. Tested groups are: 1) bone marrow-derived mature DCs; 2) MSC-CPr; 3) IFNγ-primed MSC; and 4) MSC-IPr. A) Representative flow-cytometry for the detection of the SIINFEKL/MHCI complex following pulsing with the SIINFELK peptide (positive control) or the detection of the OVA/MHCI complex following pulsing with the OVA protein. Complex detection was conducted at various time points to detect maximal activity. Black histograms represent the population in question without peptide/protein pulsing (t=0). The vertical dotted line represents the threshold level according to non-pulsed controls. B) Assessment of IFNγ production from OT-1 CD8 T cells cultured with bone-marrow derived mature DCs (dark bar) or MSC-IPr ( ) pulsed with the SIINFEKL peptide or the OVA protein for 9 or 24 hrs. For both A and B, n=5/group with *P<0.05, **P<0.01 and ***P<0.001.

FIG. 7 shows reduced tumour growth and increased survival by treating with a prophylactic vaccine comprising MSC-IPr. A) Prophylactic vaccination using OVA (mice SC- challenged. Prophylactic vaccination against EG.7 lymhoma cells. C57BL/6 mice were vaccinated using OVA-pulsed dendritic cells (DC), MSC (non-primed, non-modified MSC), IFNγ-primed MSC (γMSC), or MSC-IPr. Non-immunized animals injected with the EG.7 tumor cells are shown as control (c). B) Prophylactic vaccination using OVA (mice IV-challenged). Same experiment as A but challenged using the IV route (Control mice (c), dendritic cells (DC), and MSC-IPr). C) Prophylactic vaccination against EL4 tumor using EL4 lysate (mice SC-challenged) (Control mice (c), dendritic cells (DC), and MSC-IPr). D) Prophylactic vaccination against B16 tumors using B16F0 lysate (mice IV-challenged). (Control mice (c), dendritic cells (DC), and MSC-IPr).

FIG. 8 shows efficacy of therapeutic or treatment vaccine comprising MSC-IPr. A) Schedule used for the EG.7 therapeutic vaccine. Tumor implantation at day 0; vaccine administration at week 1.5 and 2.5. B) Therapeutic vaccination using MSC-IPr pulsated with tumor lysate. Control mice (C), dendritic cell vaccine (DC), IFNγ-primed MSC (yMSC) or MSC-IPr. At the end of the experiment, the EG.7 tumor was isolated, digested and tested for OVA expression by western blot. 1) OVA protein-positive control; 2) EL4 lysate-no OVA; 3) in vitro cultured EG.7 (eg. EL4-expressing OVA); and 4) EG.7 isolated from tumor masses at week 7. C) Kaplan-meier survival curves of panel B.

FIG. 9 shows efficacy of combination treatment with therapeutic or treatment vaccine comprising MSC-IPr, and a checkpoint inhibitor (anti-PD-1 antibodies). Control mice (C), MSC-IPr and isotype control for the MSC-IPr (IPr/-PD1), or MSC-IPr and anti-PD1 (IPr/+PD1). A) Schedule used for the EG.7 therapeutic vaccine. Tumor implantation at day 0; vaccine administration at week 1 and 2; and treatment with anti-PD-1 antibodies at week 2 and 3. D) Schedule used for the EG.7 therapeutic vaccine. Tumor implantation at day 0; vaccine administration at week 0.5 and 1.5; and treatment with anti-PD-1 antibodies at week 1 and 2. B, C, E, and F) Same experiment as FIG. 8, B and C, except the vaccine was either prior to anti-PD-1 antibodies. B and C) Therapeutic vaccination using MSC-IPr pulsated with tumor lysate followed by treatment with anti-PD-1 antibodies, for a large size tumour. n=5/group **P<0.01 and ***P<0.001. E and F) Therapeutic vaccination using MSC-IPr pulsated with tumor lysate followed by treatment with anti-PD-1 antibodies, for a small size tumour. n=10/group with **P<0.01 and ***P<0.001.

FIG. 10 shows the increase in foot pad difference following subcutaneous injection with Leishmania major parasite of: vaccinated mice (vaccinated with MSC-IPr that were pulsed with the parasite lysate) or control mice (injected with MSC-CPr). A) Vaccination schedule: vaccination with MSC-IPr treated with the parasite lysate at weeks −4 and −2, and infection with the parasite at week 0. B) The foot pad increase in of mice vaccinated with MSC-CPr is represented by the top line. The foot pad increase of mice vaccinated with MSC-IPr is represented by the bottom line. C) Pictures of the foot paws of a na-ve mouse (no vaccination and not infected with Leishmania major parasite), control mouse (injected with MSC-CPr, followed by infection with Leishmania major parasite), and vaccinated mouse (vaccinated with MSC-IPr that were pretreated with the parasite lysate, followed by infection with the Leishmania major parasite).

### DETAILED DESCRIPTION

The present disclosure relates to host cells that have been genetically modified. In some embodiments, the genetically modified host cells exhibit the properties and/or functions associated with an antigen-presenting cell (APC). In some embodiments, the genetically modified host cells have enhanced properties and/or functions associated with an APC as compared to host cells without the genetic modification.

In some embodiments, genetically modified host cells are provided that overexpress polypeptides having immunoproteasome activity. In some embodiments, genetically modified host cells are provided that has an increased expression of polypeptides having immunoproteasome activity as compared to host cells without the genetic modification. In some embodiments, genetically modified host cells are provided that has heterologous expression of polypeptides having immunoproteasome activity as compared to host cells without the genetic modification. As referred to herein, when a host cell is qualified has “genetically modified” or as being “genetically engineered”, it is understood to mean that it has been manipulated to either add at least one or more heterologous or exogenous nucleic acid residue and/or remove at least one endogenous (or native) nucleic acid residue. The genetic manipulations did not occur in nature and is the results of in vitro manipulations of the host cell. When the genetic modification is the addition of an heterologous nucleic acid molecule, such addition can be made once or multiple times at the same or different integration sites. When the genetic modification is the modification of an endogenous nucleic acid molecule, it can be made in one or both copies of the targeted gene.

As used herein, a “host cell” is any cell that is capable of being genetically modified. In some embodiments, a host cell is a somatic cell. Examples of somatic cells include, but are not limited to: epithelial cells, nerve cells, muscle cells, connective tissue cells, immune cells, blood cells, bone cells, fat cells, endothelial cells, pancreatic cells, ectoderm cells, mesoderm cells, and endoderm cells.

As used herein, “overexpression” refers to making multiple copies of a protein or gene in a cell, or increasing the expression level of a protein or gene relative to endogenous or native expression levels by genetic modification or heterologous expression of the protein.

When expressed in a host cell, the polypeptides described herein are encoded on one or more heterologous nucleic acid molecule. The term “heterologous” when used in reference to a nucleic acid molecule (such as a promoter or a coding sequence) refers to a nucleic acid molecule that is not natively found in the host cell. “Heterologous” may also include a native coding region, or portion thereof, that is removed from the source organism and subsequently reintroduced into the source organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or protein) that is derived from a source other than the endogenous source. Thus, for example, an heterologous element could be derived from a different strain of host cell, or from an organism of a different taxonomic group (e.g., different domain, kingdom, phylum, class, order, family genus, or species, or any subgroup within one of these classifications). The term “heterologous” is also used synonymously herein with the term “exogenous”.

The heterologous nucleic acid molecules of the present disclosure comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecule can be introduced in the host cell using a vector. In some embodiments, a “vector” is a “plasmid” introduced into the host cell by transduction, such as viral transduction. Transduction is a publically known tool used by molecular biologists to stably introduce a foreign gene into a host cell's genome.

In some embodiments, the polypeptides described herein are introduced by viral transduction. In other embodiments, the polypeptides described herein are introduced by CRISPR. Other methods of gene editing can also be used.

In some embodiments, the polypeptides described herein are heterologous relative to the host cells. In some embodiments, the polypeptides described herein are chimeric. As used herein, a “chimeric” protein is derived from two or more sources. For example, the chimeric may have a first polypeptide or domain derived from a first source, which is complexed with or fused to a second polypeptide or domain derived from a second source.

In some embodiments, the host cell is a stem cell. In the context of the present disclosure, the term “stem cell” refers to cells that can differentiate into other types of cells and can also divide in self-renewal to produce more of the same type of stem cells. As used herein, stem cells include “progenitor cells”. A progenitor cell has a tendency to differentiate into a specific type of cell, and have been induced to start differentiating into its target specialized cell. Progenitor cells can divide only a limited number of times to produce more of the same type of progenitor cells. As used herein, stem cells include “pluripotent stem cells”. A pluripotent stem cell is a cell that propagates indefinitely and is able to specialize into an ectoderm cell, an endoderm cell, or an mesoderm cell.

Stem cells include “embryonic stem cells” which are isolated from the inner cell mass of blastocysts in early embryonic development, and “adult stem cells”, which are found in various tissues of fully developed mammals. Adult stem cells include induced pluripotent stem cells (iPSCs) which are adult cells that have been converted into pluripotent stem cells.

In some embodiments, the stem cells described herein are obtained or collected from bone marrow, adipose tissue, umbilical cord blood or tissue, Wharton's Jelly, endometrium, placenta, brain, peripheral blood, blood vessels, skeletal muscle, skin, teeth, heart, gut, liver, ovarian epithelium, or testis. In the context of human stem cells, in some embodiments the stem cells are obtained or collected from the same individual for which treatment with the stem cell is intended.

In some embodiments, the stem cells of the present disclosure are mammalian stem cells that have been obtained or derived from a mammalian source. In one embodiment, the stem cell is a human stem cell. In preferred embodiments, the stem cell is derived from the same patient for whom treatment is intended.

### Modified Mesenchymal Stem Cells

In preferred embodiments, the stem cell is a mesenchymal stem cell (MSC). There are various advantages to using MSCs as candidate antigen-presenting cells, including MSCs i) can be easily harvested from many places such as bone marrow, adipose, Umbilical cord blood or tissue, Whartons Jelly, endometrium, or placenta, ii) display rapid in vitro proliferation, iii) require simple culture conditions, iv) exhibit low senescence through multiple passages, v) are highly permissive to gene modification, and vi) exhibit distinct in vivo migration capabilities. Mesenchymal stem cells is used for tissue repair and wound healing due to their in vivo migration and differentiation abilities. Furthermore, MSCs can display remarkable immunomodulatory properties.

However, these immune functions are influenced by surrounding pro-inflammatory cytokines, which is why MSCs demonstrate contradictory immune-related properties. For instance, low amount of IFNγ (<25 pg/ml) triggers an antigen presenting cell (APC)-like function in MSCs whereas higher IFNγ levels correlate with MSCs switching roles as immune-suppressor cells. However, from a therapeutic point of view, IFNγ treatment of MSCs is not suitable for cellular vaccination as such treatment can also halt the APC-like function of MSCs, and/or causes expression of immune checkpoint inhibitor PD-L1 ligand (Chan et al., 2006). The PD-1 receptor is an immune checkpoint on T cells that instructs them not to attack any cell carrying PD-L1. Blocking the interaction between PD-1 and PD-L1 lets T cells attack tumor cells that produce PD-L1 as a mechanism to evade the immune system. Expression of immune checkpoint inhibitor PD-L1 ligand is known for its ability to impair cytotoxic T lymphocyte (CTL) effector function and metabolism. (Stagg et al., 2006; Chan et al., 2006; Karwacz et al., 2011; Parry et al, 2005; Patsoukis et al., 2015) Furthermore, IFNγ-treated MSCs elicited low levels of IFNγ from responding T cells clearly indicating inefficient CTL priming.

In the context of the present disclosure, genetically modified mesenchymal stem cells are provided to enhance the APC-like function of MSCs. In some embodiments, genetically modified mesenchymal stem cells are provided for function as an antigen-presenting cell in vivo or ex vivo. In some embodiments, genetically modified mesenchymal stem cells are provided having immunomodulatory properties that is independent of IFNγ treatment. Such genetically modified MSC do not require IFNγ treatment to exhibit APC-like function. In some embodiments, genetically modified mesenchymal stem cells are provided overexpressing or having heterologous expression of the one or more polypeptides for the expression of an enzyme having immunoproteasome activity as described herein. In one embodiment, the genetically modified mesenchymal stem cells have one or more heterologous nucleic acid molecule encoding one or more subunits of the immunoproteasome. In one embodiment, the genetically modified mesenchymal stem cells have one or more heterologous nucleic acid molecule encoding all three β1i, β2i, and β5i subunits of IPr.

In some embodiments, the genetically modified host cells, such as mesenchymal stem cells, described herein exhibit immune-activation and pro-inflammatory response. In some embodiments, the genetically modified mesenchymal stem cells described herein elicits improved immune response from T-cells with limited expression of immune checkpoint receptors.

lmmunoproteasome

Proteasomes are protein complexes which degrade proteins by proteolysis, a chemical reaction that breaks peptide bonds (Groettrup et al., 2010; Glickman et al., 2010; Asher et al., 2006). All eukaryotic cells express the constitutive proteasome (CPr). The three proteolytic CPr subunits (β1, β2, and β5) have different preferences for peptide cleavage sites (Jayarapu et al., 2007; Khan et al., 2001; Heink et al., 2005; Toes et al., 2001; Chapiro et al., 2006). On the other hand, immunoproteasome (IPr) or IPr complex is a protein complex of multiple associated polypeptide chains (including β1i, β2i, and β5i) that is constitutively expressed in APCs (e.g. dendritic cells, macrophages and B cells) under steady state conditions (Jayarapu et al., 2007; Khan et al., 2001; Heink et al., 2005; Toes et al., 2001; Chapiro et al., 2006). Most cells can de novo assemble the IPr following IFNγ exposure by replacing the CPr β subunits with the three IFNγ-inducible homologues β1i, β2i, and β5i which are associated with IPr (Jayarapu et al., 2007; Khan et al., 2001; Heink et al., 2005; Toes et al., 2001; Chapiro et al., 2006).

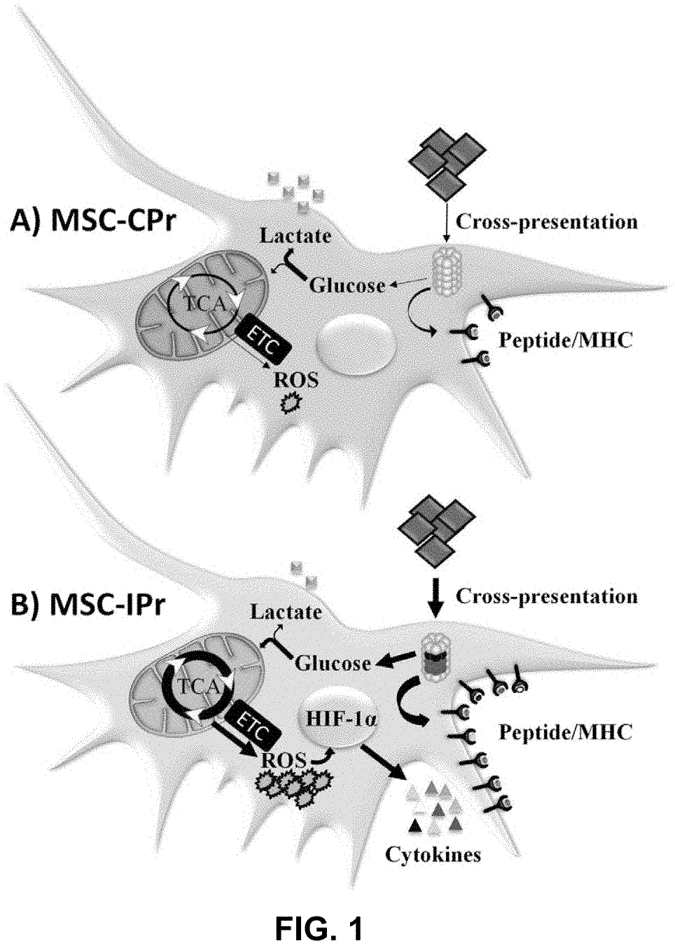

IPr function is crucial for a cell to exhibit immunomodulatory properties, since IPr readily degrades proteins, including antigen proteins, and generates peptides fitting snugly in major histocompatibility (MHC) I grooves leading to stable peptide-MHCI complexes. In turn stable peptide-MHCI complexes elicit CTL response which is necessary for developing an immune response against an antigen. As summarized in FIG. 1, in contrast to a MSC expressing CPr (MSC-CPr), IPr overexpression in MSCs (MSC-IPr) triggers enhanced extracellular antigen uptake, which leads to potent cross-presentation. In parallel, MSC-IPr exhibits a superior glucose uptake, which is metabolized mainly by oxidative phosphorylation due to AMPK activation. As a result, ROS is produced, which leads to HIF-1α stabilization consequently resulting in enhanced pro-inflammatory cytokine production.

As used herein, “antigen” refers to a toxin or other foreign substance which induces an immune response in the body by. The term antigen includes antigen proteins which are proteolysed into peptide fragments that are capable of eliciting an immune response by forming immunogenic or stable MHC or human leukocyte antigen (HLA) complexes. The term antigen also includes peptide fragments of proteins from endogenous sources, but which are not normally expressed under native conditions (for example, proteins expressed by cancer cells).

As used herein, an “immune response” refers to interactions with or the generation of a response from immune cells such as T cells, B cells, helper T-cells, NK cells, monocytes, macrophages, plasma cells, neutrophils, platelets, and/or dendritic cells for destruction of an antigen.

In the context of the present disclosure, genetically modified host cells are provided to overexpress or having heterologous expression of one or more polypeptides for the expression of an enzyme having immunoproteasome activity. In some embodiments, the enzyme having immunoproteasome activity is the IPr complex, a variant thereof or a fragment thereof. In some embodiments, the one or more polypeptides are one or more subunits of the IPr complex, a variant thereof or a fragment thereof. In some embodiments, genetically modified host cells are provided to overexpress or having heterologous expression of one, two, or all of the submits of the IPr complex, a variant thereof or a fragment thereof. In some embodiments, genetically modified host cells are provided that overexpress or have heterologous expression of one, two, or all of subunits β1i, β2i, and β5i.

In one embodiment, the genetically modified host cells overexpress or have heterologous expression of subunit β1i, β2i, or β5i. In one embodiment, the genetically modified host cells overexpress or have heterologous expression of submits β1i, and β2i. In one embodiment, the genetically modified host cells overexpress or have heterologous expression of submits β2i, and β5i. In one embodiment, the genetically modified host cells overexpress or have heterologous expression of submits β1i, and β5i. In one embodiment, the genetically modified host cells overexpress or have heterologous expression all submits β1i, β2i, and β5i.

In some embodiments, the β1i, β2i, and β5i subunits are human β1i (for example, having amino acid sequence of SEQ ID NO: 3, or encoded by nucleic acid sequence of SEQ ID NO: 2), β2i (for example, having amino acid sequence of SEQ ID NO: 5, or encoded by nucleic acid sequence of SEQ ID NO: 4), and β5i (for example, having amino acid sequence of SEQ ID NO: 7, or encoded by nucleic acid sequence of SEQ ID NO: 6) subunits, or variants or fragments thereof.

In other embodiments, the β1i, β2i, and β5i subunits are mouse β1i (for example, having amino acid sequence of SEQ ID NO: 12, or encoded by nucleic acid sequence of SEQ ID NO: 11), β2i (for example, having amino acid sequence of SEQ ID NO: 14, or encoded by nucleic acid sequence of SEQ ID NO: 13), and β5i (for example, having amino acid sequence of SEQ ID NO: 16, or encoded by nucleic acid sequence of SEQ ID NO: 15) subunits.

In some embodiments, the β1i, β2i, and β5i subunits are mammalian β1i, β2i, and β5i. For example, the β1i, β2i, and β5i subunits are canine, feline, bovine, or porcine β1i, β2i, and β5i.

The one or more polypeptides for expressing the enzyme having immunoproteasome activity include variants of the β1i, β2i, and β5i subunits of any one of SEQ ID NOs: 3, 5, 7, 12, 14, or 16 (also referred to herein as subunit variants). A subunit variant comprises at least one amino acid difference (substitution or addition) when compared to the amino acid sequence of the subunits of any one of SEQ ID NOs: 3, 5, 7, 12, 14, or 16. The subunit variants for complexes that exhibit immunoproteasome activity. The subunit variants also have at least 70%, 80%, 85%, 90%, 95%, 96%, 97%, 98% or 99% identity to the amino acid sequence of any one of SEQ ID NOs: 3, 5, 7, 12, 14, or 16. The term “percent identity”, as known in the art, is a relationship between two or more polypeptide sequences, as determined by comparing the sequences. The level of identity can be determined conventionally using known and publically available computer programs.

The subunit variant described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A subunit variant can be also be a conservative variant or an allelic variant. As used herein, a conservative variant refers to alterations in the amino acid sequence that do not adversely affect the biological functions of the immunoproteasome complex formed from the subunit variant (e.g. proteolysis). A substitution, insertion or deletion is said to adversely affect the resulting protein complex when the altered sequence prevents or disrupts a biological function associated with the immunoproteasome (e.g., proteolysis of antigen protein). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the immunoproteasome complex.

The present disclosure also provides one or more polypeptides for expressing the enzyme having immunoproteasome activity which are fragments of the β1i, β2i, and β5i subunits and subunit variants described herein. A subunit fragment comprises at least one less amino acid residue when compared to the amino acid sequence of the β1i, β2i, and β5i subunits or subunit variants and still forms an immunoproteasome complex possessing the proteolytic activity associated with the full-length subunits. The subunit fragments can also have at least 10%, 20%, 30%, 40%, 50%, 60%, 70%, 80%, 85%, 90%, 95%, 96%, 97%, 98% or 99% identity to the amino acid sequence of any one of SEQ ID NO: 3, 5, 7, 12, 14, or 16. The subunit fragment can be, for example, a truncation of one or more amino acid residues at the amino- terminus, the carboxy terminus or both terminus of the he β1i, β2i, and β5i subunits or subunit variants. Alternatively or in combination, the fragment can be generated from removing one or more internal amino acid residues.

The one or more polypeptides of the present disclosure for the expression of an enzyme having immunoproteasome activity is encoded in one or more heterologous nucleic acid molecule. In some embodiments, each of the one or more polypeptides are encoded in different a heterologous nucleic acid molecule. In some embodiments, two of the one or more polypeptides are encoded in the same heterologous nucleic acid molecule. In other embodiments, all of the one or more polypeptides are encoded in the same heterologous nucleic acid molecule.

In some embodiments, the β1i, β2i, and β5i subunits described herein are encoded in separate heterologous nucleic acid molecules. In one embodiment, the β1i, β2i, and β5i subunits described herein are encoded in one heterologous nucleic acid molecule, and transduced using a multicistronic vector.

In one embodiment, the multicistronic vector comprises a nucleic acid sequence encoding the β1i, β2i, and β5i subunits, and a 2A sequence separating each subunit. As used herein, a “2A sequence” refers to short sequences that result in the ribosome skipping the synthesis of a peptide bond at the C-terminus of a 2A element, leading to separation between the end of the 2A sequence and the next peptide downstream. In some embodiments, the “cleavage” occurs between the Glycine and Proline residues found on the C-terminus meaning the upstream cistron will have a few additional residues added to the end, while the downstream cistron will start with the Proline. Example 2A sequences include T2A sequence (SEQ ID NO: 9), P2A sequence (SEQ ID NO: 17), E2A sequence (SEQ ID NO: 18), or F2A sequence (SEQ ID NO: 18).

In some embodiments, the multicistronic vector comprises a kozak sequence at the beginning to enhance gene expression.

In some embodiments, the multicistronic vector comprises a nucleic sequence of SEQ ID NO: 1, 10, or complement thereof. As used herein, “complements” refer to nucleic acid sequences obtained from complementary base-pair matching.

Uses of and Treatments using Genetically Modified Host Cells

In the context of the present disclosure, the genetically modified host cells provided herein are intended for enhancing a patient's immune response to a target. As used herein, a “target” refers to an antigen (such as a particle, a whole cell, fragments thereof, or immunogenic peptides) against which the cell-based vaccine is designed to elicit an immune response against. Example targets include but are not limited to, a virus, a bacteria a parasite, a viral protein, a bacterial protein, a parasitic protein, a fragment of a virus, a fragment of a bacterial cell, a fragment of a parasitic cell, cancer cells, a cancer-specific protein, and/or a fragment of a cancer cell. In some embodiments, the genetically modified host cells provided herein are contacted with a target sample, such as a viral lysate, a bacterial lysate, a parasitic lysate, and/or a tumor lysate.

Example viruses include but are not limited to viruses of the following genus: Alphacoronavirus, Alphapapillomavirus, Alphatorguevirus, Alphavirus, Arenavirtis, Betacoronavirus, Cardiovirus, Cosavirus, Cytomegalovirus, Deltavirus, Dependovirus, Ebolavirus, Enterovirus, Erythrovirus, Flavivirus, Hantavirus, Henipavirus, Hepacivirus, Hepatovirus, Hepevirus, Influenzavirus Kobuvirus, Lentivirus, Lymphocryptovirus, Lyssavirus, Mamastrovirus, Marburgvirus, Mastadenovirus, Molluscipoxvirus, Morbilivirus, Mupapillomavirus, Nairovirus, Norovirus, Orthobunyavirus, Orthohepadnavirus, Orthopneumovirus, Orthopoxvirus, Parapoxvirus, Pegivirus, Phlebovirus, Polyomavirus, Respirovirus, Rhadinovirus, Roseolovirus, Rotavirus, Rubulavirus, SaUvrus, Sapovirus, Seadornavirus, Simplexvirus, Spumavirus, Thogotovirus, Torovirus, Varicellovirus, and Vesiculovirus.

Example bacteria include but are not limited to bacteria belonging to Brucella, Burkholderia, Campylobacter, Chlamydia, Chlamydophila, Ehrlichia, Francisella, Legionella, Listeria, Mycobacterium, Neisseria, Nocardia, Pseudomonas, Rickettsia, Salmonella, Shigella, Staphylococcus, Streptococcus, and Yersinia.

Example parasites include but are not limited to Cryptosporidium spp., Giardia intestinalis, Cyclospora cayetanensis, and Toxoplasma gondii; roundworms such as Trichinella spp. and Anisakis spp.; and tapeworms such as Diphyllobothrium spp. and Taenia.

In some embodiments, the genetically modified host cells are directly administered to a subject in order to elicit or enhance an immune response in vivo or ex vivo. The genetically modified host cells can be directly administered to a target location in a patient as desired. For example, the genetically modified host cells are administered by intravenous, intramuscular, intraarterial, intracoronary, intramyocardial, intrathecal, or intranasal administration.

In some embodiments, the genetically modified host cells are pretreated or pulsed with the target prior to administration or use. As used herein, “pretreated or pulsed” refers to bringing the genetically modified host cells in contact with the target, under conditions that allows the genetically modified host cells to lyse the target. Pretreated or pulsed genetically modified host cells can subsequently present the target in the form of an antigen/MHC complex.

In one embodiment, the genetically modified host cell described herein enhances the digestion of an antigen protein. In one embodiment, the genetically modified host cell described herein enhances the cross-presentation of antigen protein. In yet other embodiments, the genetically modified host cell described herein enhances both the digestion and cross-presentation of antigen protein.

In the context of the present disclosure, the genetically modified host cells provided herein are intended for use in making vaccines. In some embodiments, the vaccine is a virus, bacteria, or parasite vaccine. In some embodiments, the vaccine is a cancer vaccine. In one embodiment, the cancer vaccine is patient-specific. In one embodiment, the cancer vaccine is a prophylactic vaccine. In other embodiments, the cancer vaccine is a therapeutic vaccine. In some embodiments, the vaccines comprise genetically modified host cells described herein which have been pretreated with a target.

In some embodiments, a vaccine comprises the pulsed or pretreated genetically modified host cells and a pharmaceutically acceptable carrier. Typically a vaccine will comprise antigen (proteins), an adjuvant, and excipients or a pharmaceutically acceptable carrier. As used herein, “pharmaceutically acceptable carriers” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, etc. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods. The pharmaceutically acceptable carrier is solid or liquid. For liquid pharmaceutically acceptable carriers, the vaccine is prepared for parenteral administration, such as, but not limited to, subcutaneous injections.

In some embodiments, the pulsed or pretreated genetically modified host cells are co-administered with a second agent. As used herein, “co-administration” of the second agent refers to administration simultaneously with the pulsed or pretreated genetically modified host cells, or following administration with the pulsed or pretreated genetically modified host cells. In one embodiment, the second agent is a cytokine. Example cytokines that enable the production of molecules to further enhance immunity include, but are not limited to interleukins such as IL-2, IL-4, IL-5, IL-6, IL-7, IL-10, IL-12, IL-13, IL-15, IFNgamma or TNF-alpha.

In some embodiments, the pulsed or pretreated genetically modified host cells are co-administered with an immune checkpoint inhibitor, in particular for cancer treatments. In one embodiment, prophylactic treatment of cancer includes treatment with the pulsed or pretreated genetically modified host cells and an immune checkpoint inhibitor. In one embodiment, the therapeutic treatment of cancer includes treatment with the pulsed or pretreated genetically modified host cells and an immune checkpoint inhibitor. Example immune checkpoint inhibitor includes, but not limited to, anti-PD1 inhibitors such as anti-PD1 antibodies, anti-CTLA4, anti-LAG3, or anti-TIM3.

In the context of the present disclosure, the genetically modified host cells provided herein are intended for identifying new peptide antigens. In some embodiments, the genetically modified host cell is exposed to a target, and subsequently peptide fragments are collected from the modified host cell. The peptide fragments are screened to identify antigenic peptide fragments.

In the context of the present disclosure, the genetically modified host cells provided herein are intended for obtaining exosomes/extracellular vesicles to be used as a cell- free vaccine for immunotherapy. In some embodiments, the genetically modified host cells provided herein are cultured in a culture medium, such as a liquid culture medium. The supernatant from the culture medium containing the product of the genetically modified host cells, which is then collected and filtered. In one embodiment, the filtrate is then concentrated. The resulting product or pellet is then used for immunotherapy. The resulting product or pellet is also used in the manufacture of a medicament for immunotherapy. The collected product from the supernatant include exosomes, microvesicles (lipid nanovesicles), and/or peptides or proteins expressed by the genetically modified host cells.

The present invention will be more readily understood by referring to the following examples which are given to illustrate the invention rather than to limit its scope.

### EXAMPLE I

### Transduction of Mesenchymal Stem Cells for Heterologous Expression of Immunoproteasome

Isolation of bone marrow-derived mesenchymal stem cells (MSCs). To generate MSCs, the femur of 6-8 weeks old female C57BL/6 mice was flushed with Alpha Modification of Eagle's Medium (AMEM) supplemented with 10% FBS, and 50 U/mL Penicillin-Streptomycin in 10 cm cell culture dish (CellStar™). After 48 hours, non-adherent cells were removed. The media was changed every 3 to 4 days. When the cells reached 80% confluency, adherent cells were detached using 0.05% Trypsin, harvested, and expanded until a homogenous population was obtained before being assessed using flow-cytometry for the expression of surface markers CD44, CD45, CD73, CD90 and CD105. The differentiation capacity of isolated MSCs was tested as previously described by Eliopoulos N. et al. (Mol Ther. 2011), the entire content of which is incorporated herein by reference.

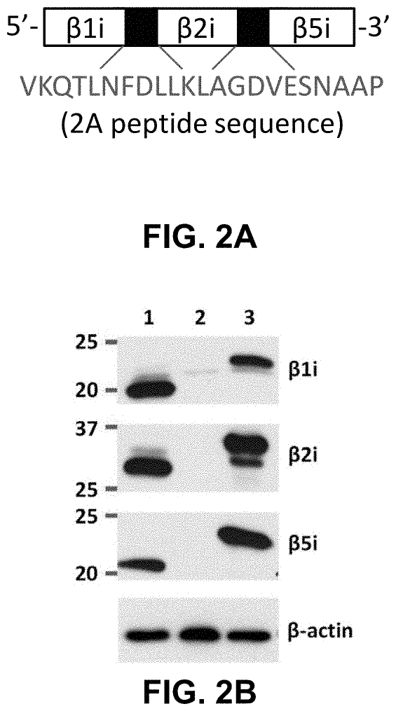

Retroviral transduction of MSCs. A construct was designed containing SEQ ID NO: 10, which comprises the cDNA of three inducible subunits of the murine immunoproteasome (β1i, β2i, and β5i), and separated by the viral T2A sequence (see FIG. 2A). The construct also contained a kozak sequence before the β1i subunit to enhance gene expression. The designed construct was then sub-cloned into the AP2 retroviral plasmid and sequenced. The AP2 construct contains the enhanced green fluorescence protein (eGFP) which serves as a marker for retroviral expression. The obtained construct was then co-transfected into the GP2-293 packaging cell line along with the VSV-G vector encoding the coating protein using Lipofectamine® (Qiagen) according to manufacturer protocol. Supernatant containing the virus was collected at 48 and 72 hours post-transfection. The supernatants were then centrifuged at 1 500 rpm for 5 min at 4° C. to remove cell debris followed by ultracentrifugation at 25 0000 rpm for 90 min at 4° C. to concentrate the virus 10 folds. Collected viruses were then aliquoted and stored at −80° C. The same steps were followed using the empty AP2 construct to generate control MSCs. For transduction, the cells were plated at 50-60% and transduced with the concentrated virus.

Protein immunoblotting. MSCs were detached using 0.05% Trypsin, collected, washed with PBS and lysed for 10 min at room temperature using Cell Lytic™ lysis buffer. Cell lysates were centrifuged for at 4° C. for 15 min at 20 000 rpm and the supernatant collected. The lysate of 10⁶ cells was dissolved in loading buffer, boiled for 5 min then loaded onto a 4-12% gradient SDS-PAGE gel. Separated proteins were transferred onto activated polyvinylidenedifluoride membrane, blocked for 1h at room temperature in Tris-buffered saline and 0.1% Tween-20 buffer (TBST) containing 5% skim milk, washed three times with TBST, then incubated with primary antibodies according to manufacturer recommendations. At the end of incubation time, the blots were washed three times with TBST followed by incubation with secondary antibodies for 1 h at room temperature. After washing three times with TBST, the proteins were revealed using enhanced chemiluminescence.

As shown in FIG. 2B, the transduction efficiency was confirmed by GFP expression and immunoblotting of the immunoproteasome (IPr) subunits. Successfully transduced MSCs were sorted and the obtained population was assessed using flow-cytometry for the expression of CD44, CD45, CD73, CD90 and CD10, which are markers associated with MSC for characterization of a stem cell as MSC.

### EXAMPLE II

### Comparison of Modified Mesenchymal Stem Cells Overexpressing Immunoproteasome to a Dendritic Cell

Generation of bone marrow-derived dendritic cells (DCs) as positive control. Mouse DCs were generated by flushing the whole marrow from mouse femur using RPM 1640 supplemented with 10% fetal bovine serum (FBS), 50 U/mL Penicillin-Streptomycin, 2 mM L-glutamine, 10 mM Hepes, 1% MEM Nonessential Amino Acids, 1 mM Sodium Pyruvate, 0.5 mM 2-Mercaptoethanol (Gibco). Plated cells were then cultured with 50 ng/ml murine GM-CSF. On days 3 and 5, media was removed and fresh media containing GM-CSF was added. On day 7, the media was replaced with fresh media containing GM-CSF and LPS from Escherichia coli O111 (1 ng/ml) to stimulate DC maturation. Mature DCs were assessed by flow-cytometry for their expression of CD11 c, CD80, CD86, MHCII and MHCI. Dendritic cells have been known as being efficient at priming immune responses, and therefore are used as positive controls.

Phenotypic analysis by flow-Cytometry. To assess the expression of cell surface markers, MSCs or DCs were incubated with flow antibodies diluted according to manufacturer's instructions using the staining buffer (PBS +2% FBS) for 30 min in the dark at 4° C. After extensive washing using the staining buffer, the cells were re-suspended in 400p1 of staining buffer then analyzed by flow-cytometry.

Stable Isotope Labeling by/with Amino acids in Cell culture (SILAC) experiment. Cells were seeded at 5×10⁶ per plate then cultured for three passages in the presence of light vs. heavy amino acids. At the end of the third passage, all cells were trypsinized, lysed prior to protein digestion by trypsin. The proteome was then analyzed by LC-MS/MS.

For the SI LAC experiment done in biological triplicates; proteins were retained for further enrichment analysis if the t-test p-value from the IPr vs. control groups is smaller than 5%. R scripts, ggplot2 and clusterprofiler (PMID: 22455463) packages were used to create gene expression heatmaps, volcano plots, GSEA plots and enrichment bar plots.

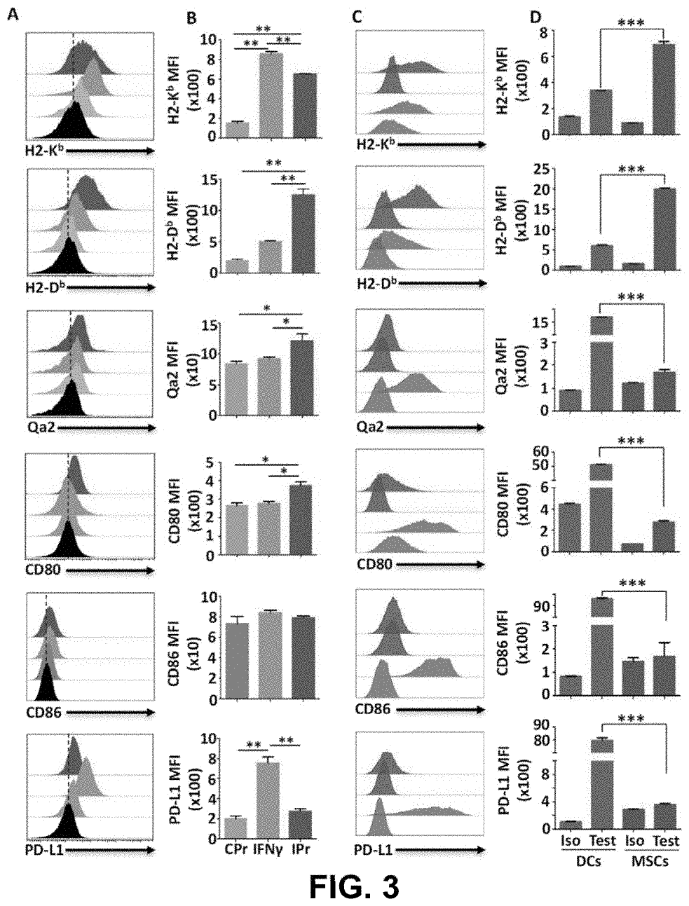

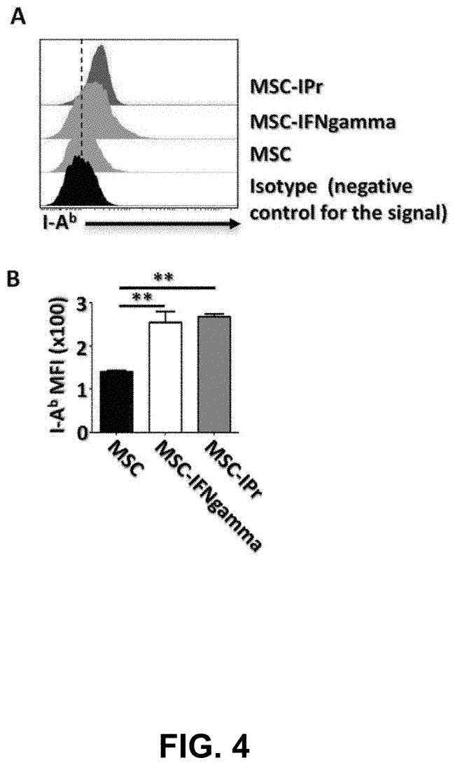

As shown in FIGS. 3 and 4, flow cytometry analysis revealed an overall enhanced MHCI (H2-Kb, H2-D^{b} and Qa2), MHCII (I-A^{b}) and CD80 expression on the surface of MSC-IPr compared to MSC-CPr in the presence or absence of IFNy. The data shows that MSC-IPr had a higher expression of MHCI molecules compared to dendritic cells (see FIG. 3, C and D). The MSC-IPr also expressed co-stimulatory molecule CD80, which is necessary for T-cell activation. The MSC-IPr also expressed MHCII molecules, which is normally only expressed on dendritic cells, macrophages and B cells (see FIG. 4). Accordingly, the modified mesenchymal stem cell overexpressing IPr expressed the intended phenotype associated with an antigen presenting cell. Not only that, the MSC-IPr had an improved phenotype expression than dendritic cells, allowing it to be more potent and longer lasting as an antigen presenting cell.

In addition, MSC-IPr did not express the immune checkpoint inhibitor PD-L1 ligand, compared to dendritic cells or IFN-gamma stimulated MSCs (see FIG. 3, A-D, last graph). As such, MSC-IPr does not impair the cytotoxic activities of T lymphocytes.

Cytokine and chemokine analysis. To assess the cytokine and chemokine profiles of DCs/MSCs, 10⁶ cells were plated in a T75 flask. The following day, the media was replaced by serum-free media and left for another 24 hrs. The supernatant was then collect then analyzed by ELISA or chemokine arrays following manufacturer's instructions.

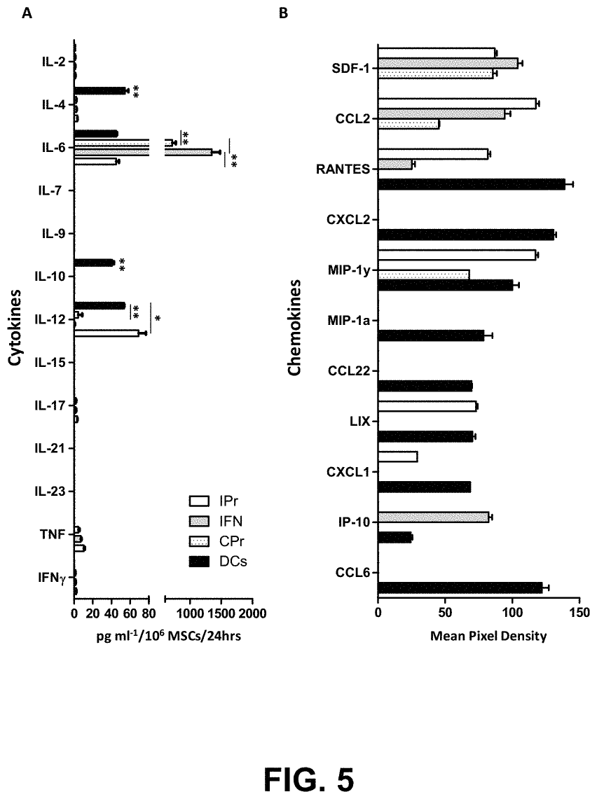

As shown in FIG. 5, the MSC-IPr showed an overall cytokine and chemokine profile that is similar to an antigen presenting cell, such as dendritic cells. Most importantly, a decrease in IL-6 and production of IL-12, which are important expression factors to antigen- presentation. This supported that MSC-IPr had the immunomodulatory characteristics of an antigen presenting cell, and in addition had an expression profile that is even better than dendritic cells.

### EXAMPLE III

### Assessing the MHC-Peptide Complex Stablity and Antigen Cross-Presentation of Modified Mesenchymal Stem Cells Pulsed with Target Antigen

Antigen presenting assay and T-cell proliferation. To evaluate their antigen cross-presentation ability, cells were seeded at 25×10³ cells per well in 24 well plate (Corning). For the control MSCy group, MSCs were pulsed with 20 ng/ml of IFNγ overnight. On the following day, the cells were washed and pulsed with the antigen of interest (5 mg/mi of ovalbumin (OVA) or 1 μg/ml of the SIINFEKL peptide (positive control)). At the end of the pulsing time, the cells were washed twice to remove excess antigen and co-cultured with 10⁶ CD8 T-cells purified from the spleen of OT-1 mouse using the CD8α⁺negative isolation kit according to the manufacturer protocol. After 72 hours, supernatants were collected and used to quantify IFNγ and IL-2 levels by ELISA. To assess antigen-specific CD8 T cells proliferation, the cells were purified from the spleen of OT-1 mice as described above and stained with CellTrace Violet according to manufacturer's instructions. Labeled cells were then co-cultured with the antigen-pulsed cell populations. Three days later, CD8 T-cells were collected and their proliferation analyzed by flow cytometry. To detect the formation of the SIINFEKL/MHCI complex, MSCs or DCs were pulsed described above and analyzed at different time points by flow cytometry using the antibody 25-D1.16.

Monitoring antigen uptake and processing. For the evaluation of OVA uptake, 4×10⁴ cells were seeded per well in 12 well plate. On the following day, 1 μg/ml conjugated OVA was added. At the end of incubation time, the cells were detached and washed with cold PBS containing 2% FBS. Fluorescence was monitored by analysing the cells on BD FACSCanto II™. For evaluating OVA processing, cells were incubated for 15-30 min at 37° C. with 10 pg/mL DQ® ovalbumin, a self-quenched conjugate of OVA that, upon proteolytic degradation, exhibits bright green fluorescence. Cells were washed, and regular media added. The signal was chased at different times. At the end of incubation time, the cells were detached and washed with cold PBS containing 2% FBS. Fluorescence was monitored by analysing the cells on BD FACSCanto II™.

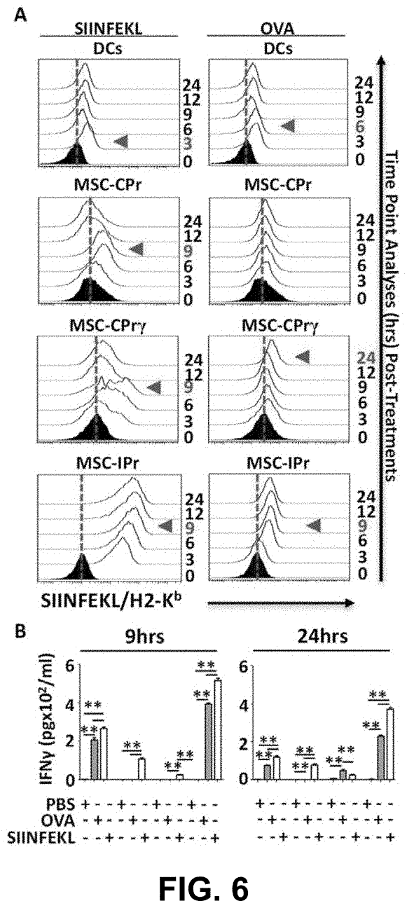

As shown in FIG. 6, pulsing the modified MSC overexpressing IPr (MSC-IPr) with a target antigen, such as OVA, resulted in the expression of OVA/MHCI complex (see panel A of FIG. 5). This experiment showed two important points. First, adding a target protein led to its processing and cell surface presentation at a much higher level than dendritic cells, (by comparing the MFI). Second, the turnover rate of the peptide-MHC complex was much lower (e.g. more stable than dendritic cells or the other MSC populations.). Even for the positive control SIINFEKL peptide pulsing (left panels of FIG. 6, A and B), the data showed higher signal intensity and lower turnover.

As shown in panel B of FIG. 6, this experiment showed that MSC cross- presented target antigens to CD8 T cells more efficiently that dendritic cells, and that irrespective of timeline (9-24hrs).

### EXAMPLE IV

### Cancer Vaccination Using Modified Mesenchymal Stem Cells Having Heterologous Immunoproteasome

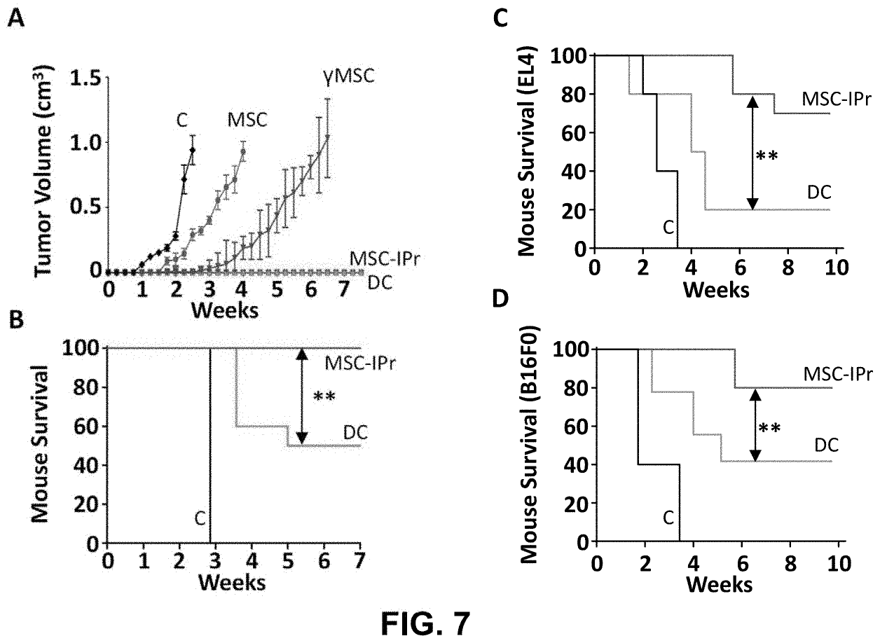

Prophylactic vaccination and tumor challenge. For prophylactic vaccination, female C57BL/6 mice (n=10/group) were IP-injected at day 0 and 14 with MSC-CPr, γMSC, MSC-IPr or DCs in the presence or absence of OVA or tumor lysate pulsing (5 mg/ml for 9 hrs). Two weeks following the second immunization, mice were subcutaneous (SC)-challenged with 10⁶ EG.7, EL4 or B16F0 tumor cells and tumor growth was then assessed using a caliper. (See FIG. 7A).

To demonstrate antigen specificity, C57BL/6 mice were immunized as detailed above and then challenged with EG.7 cells on one flank (OVA-expressing EL4 cells) vs non-OVA-expressing EL4 cells on the counter-lateral flank. Both DCs and MSC-IPr controlled EG.7 tumor growth whereas EL4 grew on the opposite flank for both treatment groups (data not shown).

To show prophylactic vaccination against EL4 or B16 tumors, MSC-IPr or DC was pulsed with EL4 or B16 tumor lysate, respectively, and then used to vaccinate mice. Tumor challenge was conducted 2 weeks later with 10⁶ tumor cells. DCs are shown in green, MSC-IPr in red and control mice in black. (See FIG. 7, panel C and D)

Immune cell infiltration analysis. Tumors were surgically removed and incubated for 90 minutes with a solution of 1.6 mg/mL collagenase type IV and 200 μg/mL DNasel in PBS. After incubation with anti-Fcγ III/II mAb (clone 2.4G2), cells were incubated for 1 hour at 4° C. with the desired antibodies or proper isotypic control. Labeled cells were subsequently analyzed by flow-cytometry (data not shown).

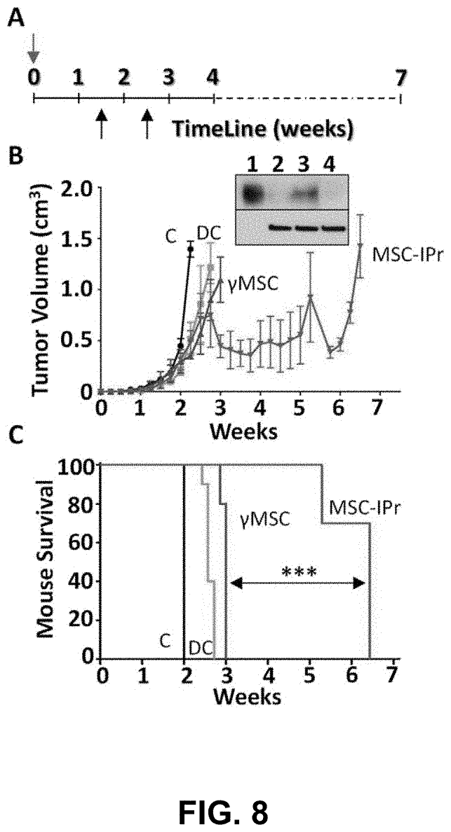

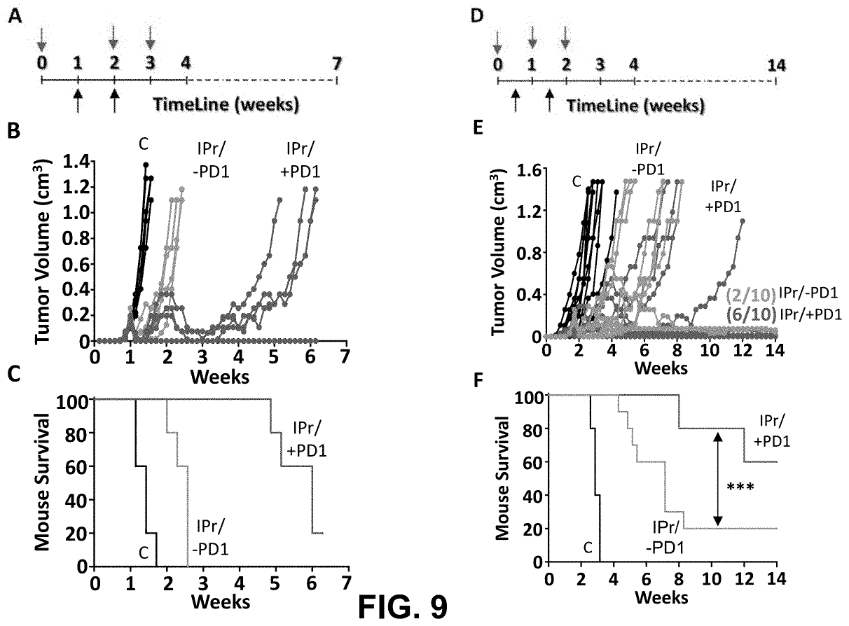

For therapeutic vaccination, female C57BL/6 mice (n=10/group) receive a SC injection of 10⁶ EG.7 cells. Following the appearance of palpable tumors (50-100 mm³), mice were IP-injected with 10⁶ OVA-pulsed DCs or MSC-IPr (two injections 1 week apart). Control animals received 10⁶ tumor cells alone or 10⁶ tumor cells followed by administration of 10⁶ non-OVA-pulsed MSC-IPr/DCs. (See FIG. 8) Treated animals were followed thereafter for EG.7 growth. Hence the PD-1 immune check inhibitor was added in combination with therapeutic vaccination MSCs-IPr for the cytotoxic T cells to function properly and target the tumour cells. For therapeutic vaccination in combination with immune-checkpoint inhibitor anti-PD-1, mice received IP-injections of the antibody or its isotype at a dose of 10 pg/injection twice. (See FIG. 9).

As shown in FIGS. 7-9, vaccination with MSC-IPr pulsated with target tumour lysate inhibited (prophylactic) or delayed (therapeutic) tumour growth and improved survival.

### EXAMPLE IV

### Parasite Vaccination Using Transduced Mesenchymal Stem Cells Having Heterologous Immunoproteasome

Leishmania vaccination. For Leishmania vaccination, Leishmania major (strain Seidman) parasite was grown at 25° C. in SDM-79 medium supplemented with 10% FBS and 5 μg of hemin /ml. For the lysate preparation, stationary phase parasites are collected and washed three times in PBS. The parasites are then re-suspended in small volume of PBS and subjected to 3 rounds of freeze/thaw cycles in liquid nitrogen and 37° C. water bath. The complete lysis of the parasites was confirmed under the microscope before proceeding with protein quantification by Bradford assay (BioRad™). The lysate was aliquoted and stored at - 80° C. until use. For vaccination, MSC-IPr were plated and the parasite lysate was added at 50 pg/mI (or different concentration and time points for cross-presentation). After 9 hrs, the cells were washed twice to remove excess lysate, counted, then re-suspended in PBS. Each vaccinated mouse received an intraperitoneal (IP) injection containing 10⁶ cells diluted in 100 μl on days 0 and 14. For negative control, each control mice received an IP injection of MSC-CPr instead.

For the challenge, stationary phase parasites (day 7-8) were counted and washed 3 times in PBS. Each mouse received subcutaneous (SC) injection of 50 μl containing 5×10⁶ parasites in the right hind foot pad.

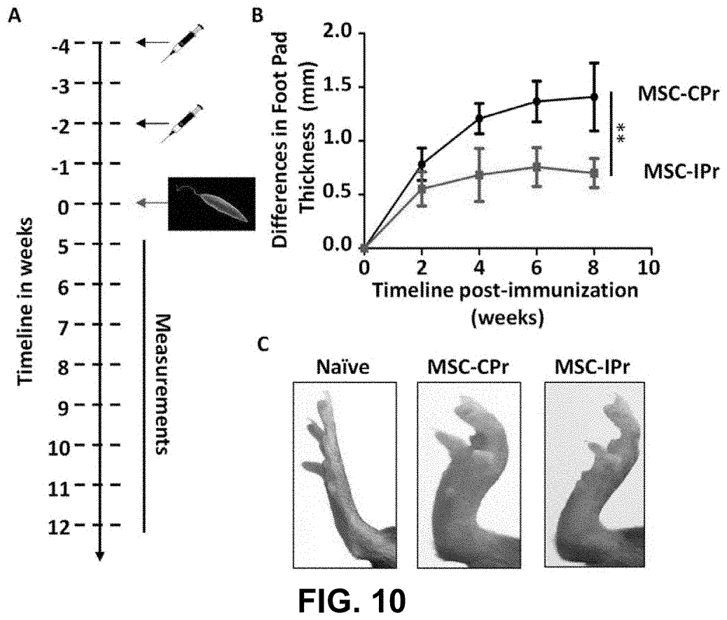

As shown in FIG. 10, mice vaccinated with MSC-IPr showed a smaller increase in foot thicket from the parasite injection, compared to mice vaccinated with MSC-CPr.

### REFERENCES

1. Appay, V., Douek, D. C. & Price, D. A. CD8+T cell efficacy in vaccination and disease. Nature medicine 2008; 14, 623-628.
2. Zhang, N. & Bevan, M. J. CD8(+) T cells: foot soldiers of the immune system. Immunity 2011; 35, 161-168.
3. Sioud M. Does our current understanding of immune tolerance, autoimmunity, and immunosuppressive mechanisms facilitate the design of efficient cancer vaccines? Scand J Immunol. 2009; 70(6):516-25.
4. Menez-Jamet J, Gallou C, Rougeot A, Kosmatopoulos K. Optimized tumor cryptic peptides: the basis for universal neo-antigen-like tumor vaccines. Ann Transl Med. 2016; 4(14):266.
5. Quezada, S. A. & Peggs, K. S. Exploiting CTLA-4, PD-1 and PD-L1 to reactivate the host immune response against cancer. British journal of cancer 2013; 108, 1560-1565.
6. Anguille, S., Smits, E. L., Lion, E., van Tendeloo, V. F. & Berneman, Z. N. Clinical use of dendritic cells for cancer therapy. The Lancet. Oncology 2014; 15, e257-267.
7. Butterfield, L. H. Cancer vaccines. BMJ (Clinical research ed.) 2015; 350, h988.
8. Lesterhuis, W. J., de Vries, I. J., Adema, G. J. & Punt, C. J. Dendritic cell-based vaccines in cancer immunotherapy: an update on clinical and immunological results. Annals of oncology: official journal of the European Society for Medical Oncology/ESMO 2004; 15 Suppl 4, iv145-151.
9. Figdor, C. G., de Vries, I. J., Lesterhuis, W. J. & Melief, C. J. Dendritic cell immunotherapy: mapping the way. Nature medicine 2004; 10, 475-480.
10. Schreiber T H, Raez L, Rosenblatt J D, Podack E R. Tumor immunogenicity and responsiveness to cancer vaccine therapy: the state of the art. Semin lmmunol. 2010; 22(3):105-12.
11. Westermann, J. et al. Cryopreservation of mature monocyte-derived human dendritic cells for vaccination: influence on phenotype and functional properties. Cancer immunology, immunotherapy: CII 2003; 52, 194-198.
12. Silk, K. M. et al. Cross-presentation of tumour antigens by human induced pluripotent stem cell-derived CD141+XCR1+dendritic cells. Gene therapy 2012; 19, 1035-1040.
13. Sachamitr, P., Hackett, S. & Fairchild, P. J. Induced pluripotent stem cells: challenges and opportunities for cancer immunotherapy. Frontiers in immunology 2014; 5, 176.
14. Gabrilovich, D. Mechanisms and functional significance of tumour-induced dendritic-cell defects. Nature reviews. Immunology 2004; 4, 941-952.
15. Pinzon-Charry, A., Maxwell, T. & Lopez, J. A. Dendritic cell dysfunction in cancer: a mechanism for immunosuppression. Immunology and cell biology 2005; 83, 451-461.
16. Eliopoulos N, Zhao J, Forner K, Birman E, Young YK, Bouchentouf M. Erythropoietin gene-enhanced marrow mesenchymal stromal cells decrease cisplatin-induced kidney injury and improve survival of allogeneic mice. Mol Ther. 2011 Nov;19(11):2072-83. doi: 10.1038/mt.2011.162. Epub 2011 Aug 16.
17. Chan J L, Tang K C, Patel A P, Bonilla L M, Pierobon N, Ponzio N M, Rameshwar P. Antigen-presenting property of mesenchymal stem cells occurs during a narrow window at low levels of interferon-gamma. Blood. 2006; 107(12):4817-24.
18. Stagg, J., Pommey, S., Eliopoulos, N. and Galipeau, J. Interferon-gamma-stimulated marrow stromal cells: a new type of nonhematopoietic antigen-presenting cell. Blood 2006; 107, 2570-2577.
19. Karwacz K, Bricogne C, MacDonald D, Arce F, Bennett C L, Collins M, Escors D. PD-L1 co-stimulation contributes to ligand-induced T cell receptor down-modulation on CD8+T cells. EMBO Mol Med. 2011; 3(10):581-92.
20. Parry R V, Chemnitz J M, Frauwirth K A, Lanfranco A R, Braunstein I, Kobayashi S V, Linsley PS, Thompson CB, Riley JL. CTLA-4 and PD-1 receptors inhibit T-cell activation by distinct mechanisms. Mol Cell Biol. 2005; 25(21):9543-53.
21. Patsoukis N, Bardhan K, Chatterjee P, Sari D, Liu B, Bell L N, Karoly E D, Freeman G J, Petkova V, Seth P, Li L, Boussiotis VA. PD-1 alters T-cell metabolic reprogramming by inhibiting glycolysis and promoting lipolysis and fatty acid oxidation. Nat Commun. 2015; 6:6692.
22. Groettrup, M., Kirk, C. J. & Basler, M. Proteasomes in immune cells: more than peptide producers? Nature reviews. Immunology 2010; 10, 73-78.
Glickman, M. H. & Ciechanover, A. The ubiquitin-proteasome proteolytic pathway: destruction for the sake of construction. Physiological reviews 82, 2002; 373-428.
23. Asher, G., Reuven, N. & Shaul, Y. 20S proteasomes and protein degradation “by default”. BioEssays : news and reviews in molecular, cellular and developmental biology. 844- 849, (2006)
24. Jayarapu, K. & Griffin, T. A. Differential intra-proteasome interactions involving standard and immunosubunits. Biochemical and biophysical research communications 358, 867-872, (2007).
25. Khan, S. et al. Immunoproteasomes largely replace constitutive proteasomes during an antiviral and antibacterial immune response in the liver. Journal of immunology (Baltimore, Md.: 1950) 2001; 167, 6859-6868.
26. Heink, S., Ludwig, D., Kloetzel, P. M. & Kruger, E. IFN-gamma-induced immune adaptation of the proteasome system is an accelerated and transient response. Proceedings of the National Academy of Sciences of the United States of America 2005; 102, 9241-9246.
27. Toes, R. E. et al. Discrete cleavage motifs of constitutive and immunoproteasomes revealed by quantitative analysis of cleavage products. The Journal of experimental medicine 2001; 194, 1-12.
28. Chapiro, J. et al. Destructive cleavage of antigenic peptides either by the immunoproteasome or by the standard proteasome results in differential antigen presentation. Journal of immunology (Baltimore, Md.: 1950) 176, 1053-1061 (2006).

Sequences

| | | |
|---|---|---|
| SEQ ID NO: 1 | DNA | Human IPr Construct full sequence |
| SEQ ID NO: 2 | DNA | Human IPr-B1i |
| SEQ ID NO: 3 | AA | Human IPr-B1i |
| SEQ ID NO: 4 | DNA | Human IPr-B2i |
| SEQ ID NO: 5 | AA | Human IPr-B2i |
| SEQ ID NO: 6 | DNA | Human IPr-B5i |
| SEQ ID NO: 7 | AA | Human IPr-B5i |
| SEQ ID NO: 8 | DNA | T2A Connnector |
| SEQ ID NO: 9 | AA | T2A Connnector |
| SEQ ID NO: 10 | DNA | Mouse IPr Construct full sequence |
| SEQ ID NO: 11 | DNA | Mouse IPr-B1i |
| SEQ ID NO: 12 | AA | Mouse IPr-B1i |
| SEQ ID NO: 13 | DNA | Mouse IPr-B2i |
| SEQ ID NO: 14 | AA | Mouse IPr-B2i |
| SEQ ID NO: 15 | DNA | Mouse IPr-B5i |
| SEQ ID NO: 16 | AA | Mouse IPr-B5i |
| SEQ ID NO: 17 | AA | P2A Connnector |
| SEQ ID NO: 18 | AA | E2A Connnector |
| SEQ ID NO: 19 | AA | F2A Connnector |

What is claimed is:

1. A genetically modified host cell having one or more heterologous nucleic acid molecule encoding one or more polypeptide for the expression of an enzyme having immunoproteasome activity, wherein the heterologous nucleic acid molecule allows for the digestion and/or cross-presentation of antigen protein by the genetically modified host cell.

2. The genetically modified host cell of claim 1, wherein the enzyme having immunoproteasome activity is a immunoproteasome, a variant thereof or a fragment thereof.

3. The genetically modified host cell of claim 1, wherein the enzyme having immunoproteasome activity is a heterologous or chimeric immunoproteasome.

4. The genetically modified host cell claim 1, wherein the one or more heterologous nucleic acid molecule each encodes one or more subunits of the enzyme having immunoproteasome activity, the one or more subunits selected from the group consisting of β1i, β3i, and β5i.

5. (canceled)

6. (canceled)

7. The genetically modified host cell of claim 4, having heterologous nucleic acid molecule encoding β1i, β2i, and β5i subunits, and a 2A sequence separating each subunit.

8. The genetically modified host cell of claim 4, wherein the β1i subunit has an amino acid sequence of SEQ ID NO: 3 or 12, a variant thereof or a fragment thereof.

9. The genetically modified host cell of claim 4, wherein the β2i subunit has an amino acid sequence of SEQ ID NO: 5 or 14, a variant thereof or a fragment thereof.

10. The genetically modified host cell of claim 4, wherein the β5i subunit has an amino acid sequence of SEQ ID NO: 7 or 16, a variant thereof or a fragment thereof.

11. The genetically modified host cell of claim 1 being a mammalian host cell.

12. The genetically modified host cell of claim 11 being a human stem cell.

13. (canceled)

14. The genetically modified host cell of claim 12 being a progenitor cell.

15. The genetically modified host cell of claim 12 being a mesenchymal stem cell.

16. The genetically modified host cell of claim 15, wherein the mesenchymal stem cell is obtained from bone marrow, adipose tissue, umbilical cord blood or tissue, Wharton's Jelly, endometrium, or placenta.

17. The genetically modified host cell of claim 15, wherein the mesenchymal stem cell is induced from a progenitor cell.

18. The genetically modified host cell of claim 12 being from embryonic stem cells or from induced pluripotent stem cells.

19.-23. (canceled)

24. A process for making vaccines, the process comprising contacting the genetically modified host cell of claim 1 with a target under a condition that promotes protein expression.

25. The process of claim 24, wherein the vaccine is a cell-based vaccine.

26. The process of claim 24 wherein the target is a virus, a bacteria, a parasite, a viral protein, a bacterial protein, a parasitic protein, a tumour sample.

27. (canceled)

28. (canceled)

29. The process of claim 26, wherein the tumour sample is a tumour lysate obtained from a patient, and wherein the vaccine is for treatment of the patient.

30.-33. (canceled)

34. A vaccine comprising the genetically modified host cell of claim 1 and a pharmaceutically acceptable carrier, wherein the genetically modified host cell has been pretreated with a target.

35. The vaccine of claim 34, wherein the target is a virus, bacteria, parasite, or a lysate thereof.

36. The vaccine of claim 34, wherein the target comprises a viral, bacterial, or parasitic protein.

37. The vaccine of claim 34, wherein the target is a tumour sample or a lysate thereof.

38. A method of treating a patient suffering from a virus, bacteria, parasite infection or cancer, comprising administering the vaccine of claim 34 to a patient in need thereof.

39. The method of claim 38, comprising co-administering with a cytokine or an interleukin.

40. The method of claim 38 for prophylactic or therapeutic treatment of cancer.

41. The method of claim 40, comprising co-administering with one or more of:
a. an immune checkpoint inhibitor;
b. a cytokine; or
c. an interleukin.

42. The method of claim 41, wherein the immune checkpoint inhibitor is an anti-PD1 inhibitor.

43.-47. (canceled)

48. A method of obtaining exosomes from the genetically modified host cell of claim 1, the method comprising:
culturing the genetically modified host cell in a culture medium;
collecting the supernatant from the culture medium; and
filtering the supernatant to collect filtrates comprising exosomes.

49. (canceled)

50. The method of claim comprising treating a patient with the collected filtrates.


SEQUENCE LISTING (19 sequences)

SEQ ID NO: 1 - DNA, 2434 bp, Artificial Sequence
  Viral transduction construct
tcgagacgcgggcggccaccatgctgcgggcgggagcaccaaccggggacttaccccgggcgggagaagtccacaccgggaccaccatcatggcagtggagtttgacgggggcgttgtgatgggttctgattcccgagtgtctgcaggcgaggcggtggtgaaccgagtgtttgacaagctgtccccgctgcacgagcgcatctactgtgcactctctggttcagctgctgatgcccaagccgtggccgacatggccgcctaccagctggagctccatgggatagaactggaggaacctccacttgttttggctgctgcaaatgtggtgagaaatatcagctataaatatcgagaggacttgtctgcacatctcatggtagctggctgggaccaacgtgaaggaggtcaggtatatggaaccctgggaggaatgctgactcgacagccttttgccattggtggctccggcagcacctttatctatggttatgtggatgcagcatataagccaggcatgtctcccgaggagtgcaggcgcttcaccacagacgctattgctctggccatgagccgggatggctcaagcgggggtgtcatctacctggtcactattacagctgccggtgtggaccatcgagtcatcttgggcaatgaactgccaaaattctatgatgaggaagggcgcgggagcctgctgacatgtggggacgtagaagaaaacccagggccaatgctgaagccagccctggagccccgagggggcttctccttcgagaactgccaaagaaatgcatcattggaacgcgtcctcccggggctcaaggtccctcacgcacgcaagaccgggaccaccatcgcgggcctggtgttccaagacggggtcattctgggcgccgatacgcgagccactaacgattcggtcgtggcggacaagagctgcgagaagatccacttcatcgcccccaaaatctactgctgtggggctggagtagccgcggacgccgagatgaccacacggatggtggcgtccaagatggagctacacgcgttatctacgggccgcgagccccgcgtggccacggtcactcgcatcctgcgccagacgctcttcaggtaccagggccacgtgggtgcatcgctgatcgtgggcggcgtagacctgactggaccgcagctctacggtgtgcatccccatggctcctacagccgtctgcccttcacagccctgggctctggtcaggacgcggccctggcggtgctagaagaccggttccagccgaacatgacgctggaggctgctcaggggctgctggtggaagccgtcaccgccgggatcttgggtgacctgggctccgggggcaatgtggacgcatgtgtgatcacaaagactggcgccaagctgctgcggacactgagctcacccacagagcccgtgaagaggtctggccgctaccactttgtgcctggaaccacagctgtcctgacccagacagtgaagccactaaccctggagctagtggaggaaactgtgcaggctatggaggtggaggaagggcgcgggagcctgctgacatgtggggacgtagaagaaaacccagggccaatgctcataggaacccccaccccgcgtgacactactcccagctcctggctgacttctagtcttctggttgaagctgcgcctttagatgacacgaccctacccacccctgtttccagcggatgcccgggcctggagcccacagaattcttccagtccctgggtggggacggagaaaggaacgttcagattgagatggcccatggcaccaccacgctcgccttcaagttccagcatggagtgattgcagcagtggattctcgggcctcagctgggtcctacattagtgccttacgggtgaacaaggtgattgagattaacccttacctgcttggcaccatgtctggctgtgcagcagactgtcagtactgggagcgcctgctggccaaggaatgcaggctgtactatctgcgaaatggagaacgtatttcagtgtcggcagcctccaagctgctgtccaacatgatgtgccagtaccggggcatgggcctctctatgggcagtatgatctgtggctgggataagaagggtcctggactctactacgtggatgaacatgggactcggctctcaggaaatatgttctccacgggtagtgggaacacttatgcctacggggtcatggacagtggctatcggcctaatcttagccctgaagaggcctatgaccttggccgcagggctattgcttatgccactcacagagacagctattctggaggcgttgtcaatatgtaccacatgaaggaagatggttgggtgaaagtagaaagtacagatgtcagtgacctgctgcaccagtaccgggaagccaatcaataatgactggatcc

SEQ ID NO: 2 - DNA, 657 bp, Homo sapiens
  CDS (1)..(657)
atgctgcgggcgggagcaccaaccggggacttaccccgggcgggagaagtccacaccgggaccaccatcatggcagtggagtttgacgggggcgttgtgatgggttctgattcccgagtgtctgcaggcgaggcggtggtgaaccgagtgtttgacaagctgtccccgctgcacgagcgcatctactgtgcactctctggttcagctgctgatgcccaagccgtggccgacatggccgcctaccagctggagctccatgggatagaactggaggaacctccacttgttttggctgctgcaaatgtggtgagaaatatcagctataaatatcgagaggacttgtctgcacatctcatggtagctggctgggaccaacgtgaaggaggtcaggtatatggaaccctgggaggaatgctgactcgacagccttttgccattggtggctccggcagcacctttatctatggttatgtggatgcagcatataagccaggcatgtctcccgaggagtgcaggcgcttcaccacagacgctattgctctggccatgagccgggatggctcaagcgggggtgtcatctacctggtcactattacagctgccggtgtggaccatcgagtcatcttgggcaatgaactgccaaaattctatgatgag

SEQ ID NO: 3 - protein, 219 aa, Homo sapiens
MLRAGAPTGDLPRAGEVHTGTTIMAVEFDGGVVMGSDSRVSAGEAVVNRVFDKLSPLHERIYCALSGSAADAQAVADMAAYQLELHGIELEEPPLVLAAANVVRNISYKYREDLSAHLMVAGWDQREGGQVYGTLGGMLTRQPFAIGGSGSTFIYGYVDAAYKPGMSPEECRRFTTDAIALAMSRDGSSGGVIYLVTITAAGVDHRVILGNELPKFYDE

SEQ ID NO: 4 - DNA, 819 bp, Homo sapiens
  CDS (1)..(819)
atgctgaagccagccctggagccccgagggggcttctccttcgagaactgccaaagaaatgcatcattggaacgcgtcctcccggggctcaaggtccctcacgcacgcaagaccgggaccaccatcgcgggcctggtgttccaagacggggtcattctgggcgccgatacgcgagccactaacgattcggtcgtggcggacaagagctgcgagaagatccacttcatcgcccccaaaatctactgctgtggggctggagtagccgcggacgccgagatgaccacacggatggtggcgtccaagatggagctacacgcgttatctacgggccgcgagccccgcgtggccacggtcactcgcatcctgcgccagacgctcttcaggtaccagggccacgtgggtgcatcgctgatcgtgggcggcgtagacctgactggaccgcagctctacggtgtgcatccccatggctcctacagccgtctgcccttcacagccctgggctctggtcaggacgcggccctggcggtgctagaagaccggttccagccgaacatgacgctggaggctgctcaggggctgctggtggaagccgtcaccgccgggatcttgggtgacctgggctccgggggcaatgtggacgcatgtgtgatcacaaagactggcgccaagctgctgcggacactgagctcacccacagagcccgtgaagaggtctggccgctaccactttgtgcctggaaccacagctgtcctgacccagacagtgaagccactaaccctggagctagtggaggaaactgtgcaggctatggaggtggag

SEQ ID NO: 5 - protein, 273 aa, Homo sapiens
MLKPALEPRGGFSFENCQRNASLERVLPGLKVPHARKTGTTIAGLVFQDGVILGADTRATNDSVVADKSCEKIHFIAPKIYCCGAGVAADAEMTTRMVASKMELHALSTGREPRVATVTRILRQTLFRYQGHVGASLIVGGVDLTGPQLYGVHPHGSYSRLPFTALGSGQDAALAVLEDRFQPNMTLEAAQGLLVEAVTAGILGDLGSGGNVDACVITKTGAKLLRTLSSPTEPVKRSGRYHFVPGTTAVLTQTVKPLTLELVEETVQAMEVE

SEQ ID NO: 6 - DNA, 819 bp, Homo sapiens
  CDS (1)..(819)
atgctcataggaacccccaccccgcgtgacactactcccagctcctggctgacttctagtcttctggttgaagctgcgcctttagatgacacgaccctacccacccctgtttccagcggatgcccgggcctggagcccacagaattcttccagtccctgggtggggacggagaaaggaacgttcagattgagatggcccatggcaccaccacgctcgccttcaagttccagcatggagtgattgcagcagtggattctcgggcctcagctgggtcctacattagtgccttacgggtgaacaaggtgattgagattaacccttacctgcttggcaccatgtctggctgtgcagcagactgtcagtactgggagcgcctgctggccaaggaatgcaggctgtactatctgcgaaatggagaacgtatttcagtgtcggcagcctccaagctgctgtccaacatgatgtgccagtaccggggcatgggcctctctatgggcagtatgatctgtggctgggataagaagggtcctggactctactacgtggatgaacatgggactcggctctcaggaaatatgttctccacgggtagtgggaacacttatgcctacggggtcatggacagtggctatcggcctaatcttagccctgaagaggcctatgaccttggccgcagggctattgcttatgccactcacagagacagctattctggaggcgttgtcaatatgtaccacatgaaggaagatggttgggtgaaagtagaaagtacagatgtcagtgacctgctgcaccagtaccgggaagccaatcaataa

SEQ ID NO: 7 - protein, 272 aa, Homo sapiens
MLIGTPTPRDTTPSSWLTSSLLVEAAPLDDTTLPTPVSSGCPGLEPTEFFQSLGGDGERNVQIEMAHGTTTLAFKFQHGVIAAVDSRASAGSYISALRVNKVIEINPYLLGTMSGCAADCQYWERLLAKECRLYYLRNGERISVSAASKLLSNMMCQYRGMGLSMGSMICGWDKKGPGLYYVDEHGTRLSGNMFSTGSGNTYAYGVMDSGYRPNLSPEEAYDLGRRAIAYATHRDSYSGGVVNMYHMKEDGWVKVESTDVSDLLHQYREANQ

SEQ ID NO: 8 - DNA, 54 bp, Artificial Sequence
  2A connector sequence
  CDS (1)..(54)
gaagggcgcgggagcctgctgacatgtggggacgtagaagaaaacccagggcca

SEQ ID NO: 9 - protein, 18 aa, Artificial Sequence
  Synthetic Construct
EGRGSLLTCGDVEENPGP

SEQ ID NO: 10 - DNA, 2420 bp, Artificial Sequence
  Viral transduction construct
tcgagacgcgggcggccaccatggccgagccccgctctgctgaggccaccatgctgcgggcaggagcacctaccgccggctcgttccggacggaagaagtccacaccgggacaaccatcatggcagtggagtttgacgggggtgtcgtggtgggctctgattcccgggtgtcagcgggaacagcagtggtgaaccgcgtgttcgacaagctctcccctctgcaccagcacatcttctgtgccctctcaggttccgctgctgatgcccaagccatagctgacatggccgcctaccagctggagctacacgggttggagctggaggagccgcccctcgttctggctgctgcaaacgtggtgaagaacatctcctacaagtaccgtgaggacttgttagcgcatctcatagtagctggctgggaccaatgtgaggggggacaggtatatggaaccatgggagggatgctaattcgacagccctcaggcatgacccctgaggagtgccggcgtttcaccacagatgccatcactctggccatgaaccgagatggctctagtgggggtgtcatctacctggtcaccatcacagctgctggtgtggaccatcgagtcatcctgggagatgagctgccaaaattctacgatgaggaagggcgcgggagcctgctgacatgtggggacgtagaagaaaacccagggccaatgctgaagcaggcagtggaacccacaggaggcttctctttcgagaactgccagaggaatgcgtccttggaacacgtccttccgggacttcgggttcctcatgcacgcaagaccgggactaccatcgcggggcttgtgttccgagatggagtcatcctgggagcggacacgcgggccactaacgattcggttgtggcggacaaaagctgcgagaagatccacttcatcgcccctaaaatctactgctgtggggctggagtagctgcggacactgagatgactacgcggatggcagcttccaagatggaactacatgcgctgtccaccggccgtgagcctcgggtggccacggtcacccgtatcctgcgccagacgcttttccggtaccaaggccacgtgggggcatcactggtcgtgggcggggttgatttgaacggacctcagctctacgaggtgcacccacatggttcctacagccgtctgccctttactgcccttggctctggtcagggtgcagccgtggcactgttggaagaccggttccagccaaacatgacgctggaggctgcgcaagagctgttggtggaagccatcacagcagggatactgagtgacctgggctctgggggcaatgtggatgcctgtgtgatcactgcagggggtgccaagctgcagagagcattgagcacccccaccgagcctgtgcagagagctggccgttaccgatttgctcctggaaccacacctgtcctgacccgggaagtgagacccctgaccctggaacttcttgaggaaactgtgcaggccatggaggtggaagaagggcgcgggagcctgctgacatgtggggacgtagaagaaaacccagggccaatggcgttactggatctgtgcggtgccgctcgggggcagcggcccgagtgggctgccctggatgcgggaagcgggggtcgctcggacccgggacactacagtttctccgcgcaagctccggagctcgcacttccccggggaatgcagcccaccgcattcctgaggtcctttggtggtgaccaggaaaggaatgttcaaattgagatggcccacggcacaaccacactcgccttcaagttccagcatggcgtcatcgtggctgtggactccagggccactgcagggagttacattagctccttaaggatgaacaaagtgatcgagattaacccttacctgcttggcaccatgtctggttgtgcagccgactgccagtactgggagaggctgttggccaaggagtgcaggttgtattatcttcggaatggggaacgcatctccgtgtctgcagcatccaagctgctttccaacatgatgctgcagtaccgggggatgggcctctccatgggcagcatgatctgtggctgggacaagaagggaccaggactttactacgtagatgacaatgggactcggctctcgggacagatgttttccactggcagcgggaacacctatgcctatggggtgatggacagtggttaccggcaggacctcagtcctgaagaggcctacgaccttggccgcagagctattgcttatgctacccacagagacaactattctggaggagtcgtcaacatgtaccacatgaaggaagacggttgggtgaaagtggagagttccgatgtcagtgacctgctgtacaagtacggagaggccgctctgtgatggctgactggatcc

SEQ ID NO: 11 - DNA, 597 bp, Mus musculus
  CDS (1)..(597)
atgctgcgggcaggagcacctaccgccggctcgttccggacggaagaagtccacaccgggacaaccatcatggcagtggagtttgacgggggtgtcgtggtgggctctgattcccgggtgtcagcgggaacagcagtggtgaaccgcgtgttcgacaagctctcccctctgcaccagcacatcttctgtgccctctcaggttccgctgctgatgcccaagccatagctgacatggccgcctaccagctggagctacacgggttggagctggaggagccgcccctcgttctggctgctgcaaacgtggtgaagaacatctcctacaagtaccgtgaggacttgttagcgcatctcatagtagctggctgggaccaatgtgaggggggacaggtatatggaaccatgggagggatgctaattcgacagccctcaggcatgacccctgaggagtgccggcgtttcaccacagatgccatcactctggccatgaaccgagatggctctagtgggggtgtcatctacctggtcaccatcacagctgctggtgtggaccatcgagtcatcctgggagatgagctgccaaaattctacgatgag

SEQ ID NO: 12 - protein, 199 aa, Mus musculus
MLRAGAPTAGSFRTEEVHTGTTIMAVEFDGGVVVGSDSRVSAGTAVVNRVFDKLSPLHQHIFCALSGSAADAQAIADMAAYQLELHGLELEEPPLVLAAANVVKNISYKYREDLLAHLIVAGWDQCEGGQVYGTMGGMLIRQPSGMTPEECRRFTTDAITLAMNRDGSSGGVIYLVTITAAGVDHRVILGDELPKFYDE

SEQ ID NO: 13 - DNA, 819 bp, Mus musculus
  CDS (1)..(819)
atgctgaagcaggcagtggaacccacaggaggcttctctttcgagaactgccagaggaatgcgtccttggaacacgtccttccgggacttcgggttcctcatgcacgcaagaccgggactaccatcgcggggcttgtgttccgagatggagtcatcctgggagcggacacgcgggccactaacgattcggttgtggcggacaaaagctgcgagaagatccacttcatcgcccctaaaatctactgctgtggggctggagtagctgcggacactgagatgactacgcggatggcagcttccaagatggaactacatgcgctgtccaccggccgtgagcctcgggtggccacggtcacccgtatcctgcgccagacgcttttccggtaccaaggccacgtgggggcatcactggtcgtgggcggggttgatttgaacggacctcagctctacgaggtgcacccacatggttcctacagccgtctgccctttactgcccttggctctggtcagggtgcagccgtggcactgttggaagaccggttccagccaaacatgacgctggaggctgcgcaagagctgttggtggaagccatcacagcagggatactgagtgacctgggctctgggggcaatgtggatgcctgtgtgatcactgcagggggtgccaagctgcagagagcattgagcacccccaccgagcctgtgcagagagctggccgttaccgatttgctcctggaaccacacctgtcctgacccgggaagtgagacccctgaccctggaacttcttgaggaaactgtgcaggccatggaggtggaa

SEQ ID NO: 14 - protein, 273 aa, Mus musculus
MLKQAVEPTGGFSFENCQRNASLEHVLPGLRVPHARKTGTTIAGLVFRDGVILGADTRATNDSVVADKSCEKIHFIAPKIYCCGAGVAADTEMTTRMAASKMELHALSTGREPRVATVTRILRQTLFRYQGHVGASLVVGGVDLNGPQLYEVHPHGSYSRLPFTALGSGQGAAVALLEDRFQPNMTLEAAQELLVEAITAGILSDLGSGGNVDACVITAGGAKLQRALSTPTEPVQRAGRYRFAPGTTPVLTREVRPLTLELLEETVQAMEVE

SEQ ID NO: 15 - DNA, 835 bp, Mus musculus
  CDS (1)..(834)
atggcgttactggatctgtgcggtgccgctcgggggcagcggcccgagtgggctgccctggatgcgggaagcgggggtcgctcggacccgggacactacagtttctccgcgcaagctccggagctcgcacttccccggggaatgcagcccaccgcattcctgaggtcctttggtggtgaccaggaaaggaatgttcaaattgagatggcccacggcacaaccacactcgccttcaagttccagcatggcgtcatcgtggctgtggactccagggccactgcagggagttacattagctccttaaggatgaacaaagtgatcgagattaacccttacctgcttggcaccatgtctggttgtgcagccgactgccagtactgggagaggctgttggccaaggagtgcaggttgtattatcttcggaatggggaacgcatctccgtgtctgcagcatccaagctgctttccaacatgatgctgcagtaccgggggatgggcctctccatgggcagcatgatctgtggctgggacaagaagggaccaggactttactacgtagatgacaatgggactcggctctcgggacagatgttttccactggcagcgggaacacctatgcctatggggtgatggacagtggttaccggcaggacctcagtcctgaagaggcctacgaccttggccgcagagctattgcttatgctacccacagagacaactattctggaggagtcgtcaacatgtaccacatgaaggaagacggttgggtgaaagtggagagttccgatgtcagtgacctgctgtacaagtacggagaggccgctctgtgatggc

SEQ ID NO: 16 - protein, 276 aa, Mus musculus
MALLDLCGAARGQRPEWAALDAGSGGRSDPGHYSFSAQAPELALPRGMQPTAFLRSFGGDQERNVQIEMAHGTTTLAFKFQHGVIVAVDSRATAGSYISSLRMNKVIEINPYLLGTMSGCAADCQYWERLLAKECRLYYLRNGERISVSAASKLLSNMMLQYRGMGLSMGSMICGWDKKGPGLYYVDDNGTRLSGQMFSTGSGNTYAYGVMDSGYRQDLSPEEAYDLGRRAIAYATHRDNYSGGVVNMYHMKEDGWVKVESSDVSDLLYKYGEAAL

SEQ ID NO: 17 - protein, 19 aa, Artificial Sequence
  P2A connector sequence
ATNFSLLKQAGDVEENPGP

SEQ ID NO: 18 - protein, 20 aa, Artificial Sequence
  E2A connector sequence
QCTNYALLKLAGDVESNPGP

SEQ ID NO: 19 - protein, 22 aa, Artificial Sequence
  F2A connector sequence
VKQTLNFDLLKLAGDVESNPGP